(19) United States
(12) Patent Application Publication
(10) Pub. No.: US 2022/0356495 A1
(43) Pub. Date: Nov. 10, 2022

(54) BIOCONVERSION OF METHANE TO 3-HYDROXYBUTYRATE

(71) Applicant: Alliance for Sustainable Energy, LLC, Golden, CO (US)

(72) Inventors: Michael T. GUARNIERI, Denver, CO (US); Rabea JESSER, Staunton, VA (US)

(21) Appl. No.: 17/657,902

(22) Filed: Apr. 4, 2022

Related U.S. Application Data
(60) Provisional application No. 63/170,257, filed on Apr. 2, 2021.

(51) Int. Cl. C12P 7/42; C12N 1/20; C12N 15/52; C12N 15/74

(52) U.S. Cl. CPC C12P 7/42; C12N 1/205; C12Y 301/01075; C12Y 207/02007; C12Y 203/01019; C12N 15/52; C12N 15/74; C12R 2001/01

(57) ABSTRACT

Disclosed herein are methods and compositions of matter that enable the biological conversion of gaseous waste streams (CO₂, stranded natural gas, flue gas, biogas, landfill gas, etc.) to a platform chemical, 3-hydroxybutyrate, which can in turn be upgraded to fuels and polymers (e.g. polypropylene and polymers). The technology thus presents both a mechanism to valorize gaseous waste streams and establish sustainable production routes to chemicals and plastics via the overexpression of PHB depolymerase while knocking out the AACS pathway in this specific strain of methanotrophic bacteria.

### CROSS-REFERENCE TO RELATED APPLICATIONS

This application claims priority under 35 U.S.C. § 119 to U.S. provisional patent application No. 63/170,257 filed on 2 Apr. 2021, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### SEQUENCE LISTING

The instant application contains a Sequence Listing which has been submitted via EFS-web and is hereby incorporated by reference in its entirety. The ASCII copy as filed herewith was originally created on 15 Jul. 2022. The ASCII copy as filed herewith is named NREL 21-21_ST25.txt, is 14,467 bytes in size and is submitted with the instant application.

### BACKGROUND

The biological conversion of gaseous waste streams (CO₂, stranded natural gas, flue gas, biogas, landfill gas, etc.) to a platform chemicals such as 3-hydroxybutyrate, which can in turn be upgraded to fuels and polymers (e.g. polypropylene and polymers), is, hitherto fore, an unmet need for the generation of renewable commodity chemicals.

### SUMMARY

In an aspect, disclosed herein are a non-naturally occurring organism capable of converting gases selected from the group consisting essentially of CO₂, stranded natural gas, flue gas, biogas, and landfill gas to 3-hydroxybutyrate. In an embodiment, the non-naturally occurring organism is a methanotrophic bacteria. In an embodiment, the organism is genetically engineered to overexpress PHB depolymerase and also lacks an acetoacetyl-CoA synthetase (AACS) pathway.

In an aspect, disclosed herein is a method for making 3-hydroxybutyrate comprising the step of contacting a non-naturally occurring organism with gases selected from the group consisting essentially of CO₂, stranded natural gas, flue gas, biogas, and landfill gas. In an embodiment, the non-naturally occurring organism is a methanotrophic bacteria. In another embodiment, the non-naturally occurring organism is genetically engineered to overexpress PHB depolymerase and also lacks a AACS pathway.

Other objects, advantages, and novel features of the present invention will become apparent from the following detailed description of the invention when considered in conjunction with the accompanying drawings.

### BRIEF DESCRIPTION OF THE DRAWINGS

FIGS. 1A and 1B depict a schematic representation of M. trichosporium OB3b native PHB synthesis and degradation pathways (FIG. 1A) and overexpression pathways implemented in this study (FIG. 1). Surplus acetyl-CoA from the TCA/EMC or serine cycles feeds the PHB pathway. Dark blue represents M. trichosporium native genes, light blue heterologous enzymes from Clostridium butyricum, arrow colors in (FIG. 1B) represent genes overexpressed in different plasmids: light orange pIPRJ016; green pIPRJ017; purple pIPRJ029; dark orange pIPRJ032; phaA, acetyl-CoA C-acetyltransferase; phaB, acetoacetyl-CoA reductase; phaC, class I poly(R)-hydroxyalkanoic acid synthase=PHB polymerase, phaZ=polyhydroxyalkanoate depolymerase; aacS, acetoacetate-CoA ligase; bdh, 3-hydroxybutyrate dehydrogenase; ptb, phosphate butyryltransferase; buk, butyrate kinase.

FIGS. 2A, 2B, 2C and 2D depict 3-HB strain characterization and Production. (FIG. 2A) Growth curve of wild type (blue), and aacS mutant (red) and bdh mutant (green) overexpressing phaABCZ1Z2 plasmid. (FIG. 2B) Relative PHB content of aforementioned strains as determined via Nile Red staining and quantitative fluorescence (FIG. 2C) 3-hydroxybutyrate titer, and (FIG. 2D) 3-hydroxybutyrate titer as a function of growth phase (OD₆₀₀).

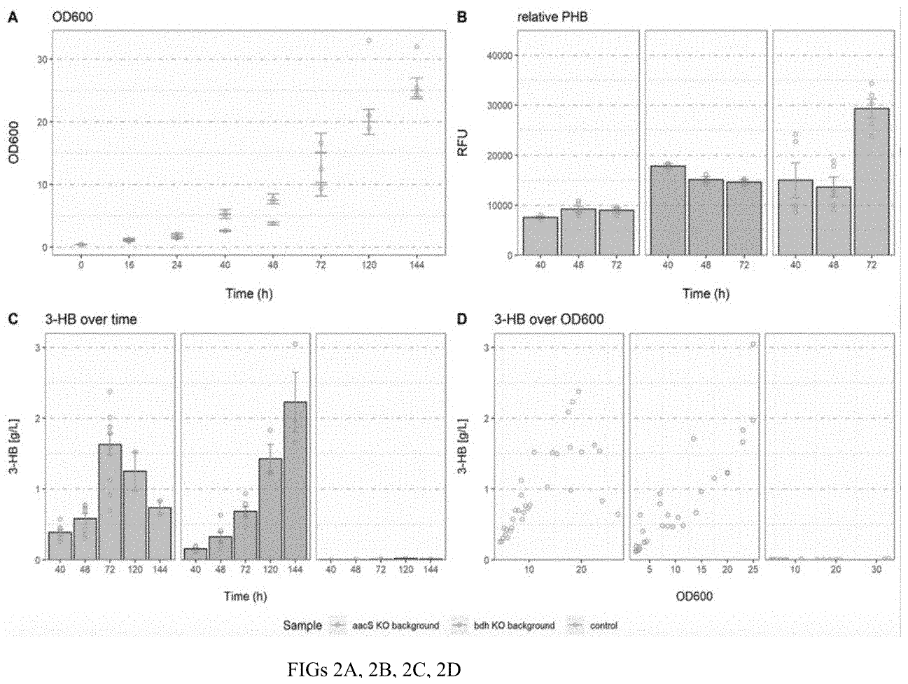

FIG. 3 depicts relative fluorescence units (RFU, corresponding to relative PHB content) from early stationary cells. Strains were cultivated in 30 ml culture volume in a sealed 160 ml serum vial supplemented with 20% CH₄ and 20% CO₂ in the headspace and the amount of cells harvested for nile red staining was normalized by OD₆₀₀. The data represent the mean+/−SEM (n=3). (WT=wild-type M. trichosporium OB3b, daacS=M. trichosporium aacS null mutant, daacS pIPRJ032=M. trichosporium aacS::pIPRJ032 (expressing M. trichosporium pha operon)).

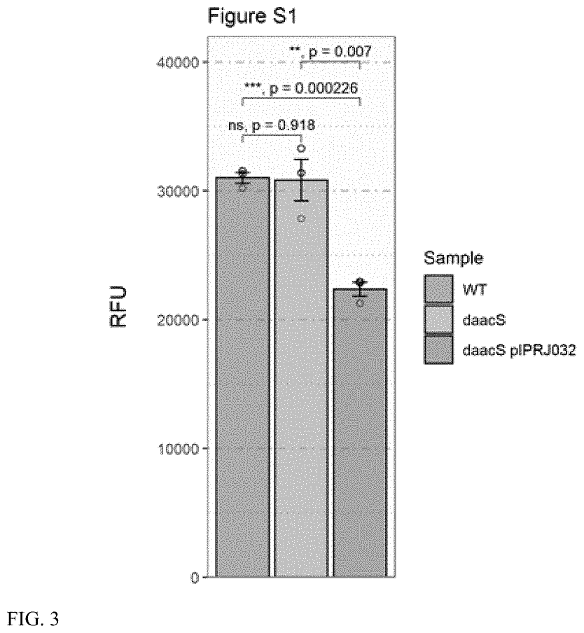

FIGS. 4A, 4B, 4C, and 4D depict a confirmation of null mutant strains: FIG. 4A—M. trichosporium OB3b aacS null mutant, FIG. 4B—M. trichosporium OB3b bdh null mutant, FIG. 4C M. trichosporium OB3b aacS/bdh null mutant, FIG. 4D—Schematic representation of the primers used to confirm null mutants. (WT=wild-type M. trichosporium OB3b, ΔaacS=M. trichosporium aacS null mutant, KO=knock-out=null mutant, p=primer, kb=kilo base pair, FRT=flippase recognition target site, GentR=Gentamicin selection marker cassette, GOI=gene of interest).

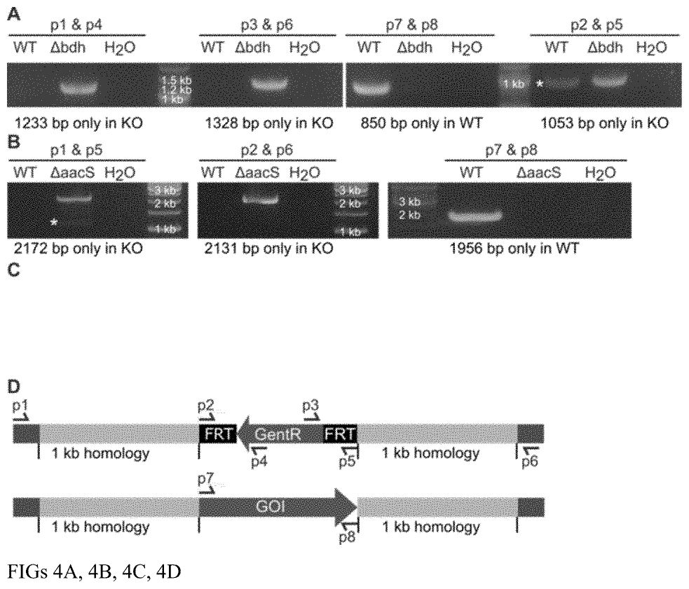

FIG. 5 depicts NAD(P)(H) levels in mid-log phase cells cultivated in continuous bioreactor (3-HB producing strain vs. wild-type (WT) control). Samples were normalized to cell number via OD600 prior to analysis; WT, wild-type M. trichosporium OB3b; ΔaacS pIPRJ032, M. trichosporium aacS null mutant expressing 3-HB operon from pIPRJ032; NAD(H), NAD++NADH levels; NADP(H)=NADP++NADPH levels; significance levels were obtained from two-sided, unpaired t-test with equal variances and a 95% confidence level (ns=not significant, *=p<0.05).

FIGS. 6A, 6B, 6C, 6D depict high fidelity PCR confirming KO background strains (Δbdh, FIG. 6A; ΔaacS, FIG. 6B; ΔaacSΔbdh, FIG. 6C), sequence identity of each candidates was confirmed by subsequent sequence analysis. Unspecific products are marked with a white asterisk. KO=knock-out, WT=wild type, p=primer, FRT=flippase recognition target, kb=kilobasepair, GentR=Gentamicin selection marker cassette, GOI=gene of interest.

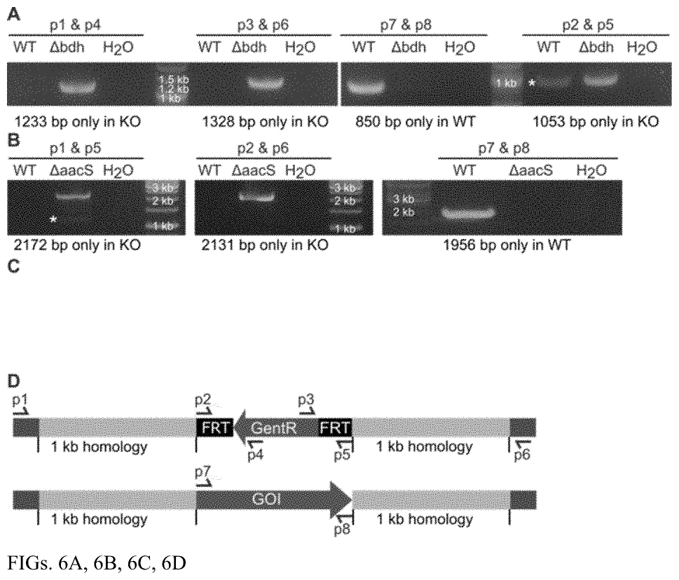

### DETAILED DESCRIPTION

Using biological systems to produce chiral compounds like 3-hydroxybutyrate (3-HB) requires lower temperatures and produces less toxic waste, fewer emissions and by-products than conventional production routes, and thus represents a sustainable alternative to conventional chemical synthesis. Hydroxycarboxylic acids, like 3-HB, can be used widely as chiral precursor for antibiotics, pharmaceuticals and fungicides, while oligomerized 3-HB has the potential to serve as a drug or antioxidant delivery vector. As an abundantly produced physiological ketone, 3-hydroxybutyrate is also thought to have an array of potential medical applications—for example as treatment for patients with tear-deficient dry eye disease. In addition, monomeric 3-HB has the potential to serve as a substrate for high-value pure polyhydroxybutyrate (PHB) or its co-polymers with superior properties compared to microbially produced PHB. Another advantage of 3-HB compared to PHB is that as an extracellular product, 3-HB can be collected straight from the medium enabling cell reuse, which is useful for relatively slow growing biomass.

Various efforts are underway to produce microbial 3-HB. The halophilic bacteria Halomonas sp. KM-1, which stores intracellular PHB under aerobic conditions and secretes the monomer under microaerobic conditions, achieved secretion of one of the highest 3-HB titers to date. In optimized culture conditions this organism produced up to 40.3 g/L (R)-3-HB in a nitrate fed-batch cultivation with 20% (w/v) glucose. Most of the efforts to produce 3-HB in metabolically engineered strains has so far been focused on the expression of heterologous pathways in model organisms such as Escherichia coli or Saccharomyces cerevisiae. A recombinant E. coli strain, overexpressing both its native thioesterase yciA and its glucose-6-phosphate dehydrogenase zwf alongside a heterologous thiolase and reductase from Halomonas boliviensis yielded the maximum titer to date in E. coli (14.3 g/L). Maximum titers in the non-conventional yeast Arxula adeninivorans were achieved by overexpressing the bacterial thiolase thl and the enantiospecific reductase (phaB) from Cupriavidus necator H16. Being able to thrive on CO₂ and sunlight, cyanobacteria have become an attractive host for the production secondary metabolites from cheap, renewable feedstock. However, with a maximum titer to date of 533.4 mg/L from a genetically engineered Synechocystis, cyanobacterial titers lack far behind those of their heterotrophic counterparts.

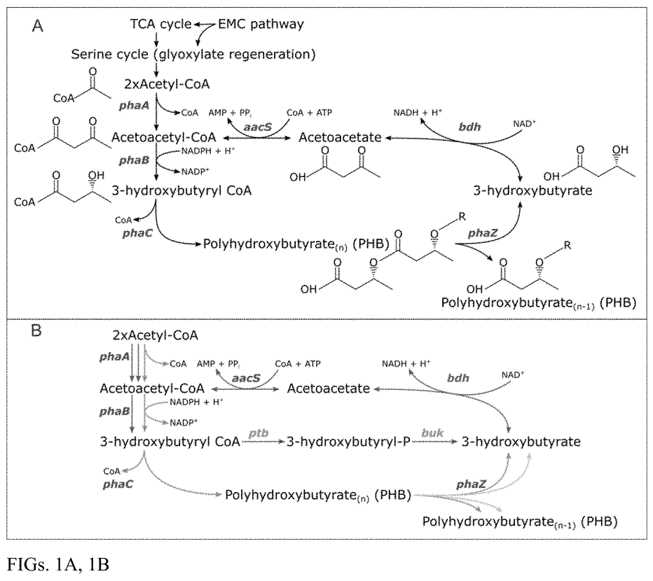

With CH₄ and CO₂ being the two most abundant anthropogenic greenhouse gases and Type II methanotrophs metabolizing both of those gases, these biocatalysts not only offer a promising greenhouse gas sink, but might also be an auspicious alternative production chassis for 3-HB. Various biochemical pathways can be envisioned to generate 3-HB levels in Type II methanotrophs. In this study, four different pathways were overexpressed, one of which yielded the highest titer of CH₄-derived acid to our knowledge to date (FIG. 1). Two pathways aimed to bypass the native PHB accumulation altogether and express either the native acetyl-CoA C-acetyltransferase (phaA) and acetoacetyl-CoA reductase (phaB) in conjunction with the native acetoacetate-CoA ligase (aacS) and 3-hydroxybutyrate dehydrogenase (bdh) or with the Clostridium butyricum (strain CFSA3989) phosphate butyryltransferase (ptb) and butyrate kinase (buk). Overexpression of the latter pathway in E. coli yielded up to 12 g/L 3-HB after 48 h from a fed-batch fermentation. The other two pathways aimed to take advantage of the native PHB machinery and overexpress either the complete PHB pathway plus the native PHB depolymerases or just the PHB depolymerase(s) by themselves. In a similar approach the Ralstonia eutropha PHA biosynthesis operon along with the an intra- or extracellular PHA depolymerase gene was transferred to E. coli yielding up to 9.9 g/L or 7.29 g/L, respectively.

In an embodiment, disclosed herein are compositions of matter comprising non-naturally occurring organisms that generate 3HB.

Generation of 3HB-Production Strains and their 3HB Production Capacity

The 3HB production capability of five different M. trichosporium OB3b strains harboring one of four different overexpression plasmids was assessed. The same basic overexpression backbone was used for all four plasmids: a promoter and terminator sequence flanking the gene of interest (GOI) arranged as an operon on an IncP broad-host-range plasmid. Since the native mxaF gene showed high expression levels in transcriptomic studies and the upstream genomic region of this gene was found to drive strong gene expression of reporter proteins, this region was used as a promoter sequence in this study. Ribosome binding sites (RBS) linking the GOI were either selected from the native genome, designed with the RBS calculator or derived from the consensus RBS AGGAGG+6 or 9 base pair downstream. All four plasmids were introduced in M. trichosporium OB3b WT background. Considering that a simple bdh null mutant in R. eutropha H16 led to a 1.67 fold increase for the mutant compared to the wild type (18 mM (=1.87 g/L) to 30 mM (=3.12 g/L)) for the mutant 3-HB in the medium, the complete PHB operon+depolymerase was also introduced into backgrounds where the flux between acetyl-CoA and 3-HB would be interrupted (aacS and bdh null mutants).

Initial analysis for 3HB production capacity in batch culture in serum vials revealed three out of five overexpression strains capable of producing measurable amounts of 3HB in the supernatant while the other two strains did not exceed background 3-HB levels. If the native PHB depolymerase (pIPRJ016) was overexpressed by itself, 3-HB levels in the supernatant only increased significantly after cells reached a stage in their growth phase when they would naturally produce PHB. Interestingly, cells overexpressing the complete PHB pathway plus the PHB depolymerase, but lacking the native acetoacetate-CoA ligase gene aacS or butyrate dehydrogenase bdh, outperformed the respective WT background in 3-HB production—indicating 3-HB reassembly capacity in the WT background. Given enough time, a similar effect was observed in the KO background indicating a switch in cellular metabolite or reductant balance (FIG. 1). Maximum 3-HB titers of up to 2 g/L were achieved after 72 h (0.028 g/(L/h) cultivation in a continuous reactor with excess nitrogen and continuous CH₄ (20%) and CO₂ (2%) supply (Table 1). Scale up in continuous bioreactor showed up to ˜13.5× increase compared to serum vial cultivation for regular nitrogen supply (Table 1). Given excess amounts of nitrogen, cultures grown in the continuous reactor produced another 4-fold more 3-3, reaching a total increase of 53× compared to serum vial cultivation.

TABLE 1
Maximum 3HB titers from M. trichosporium OB3b strains overexpressing different genomic operons.

| | | Max. 3-HB | Max. 3-HB | | |
|---|---|---|---|---|---|
| Overexpressed | genomic | titer | titer | | |
| genes | background | (mM) ± SEM | (g/L) ± SEM | Culture method | Growth phase |
| phaZ1Z2 | WT | 0.032 ± 0.00084 | 0.0033 ± 0.00010 | batch culture | after 24 h |
| | | | | | nitrogen |
| | | | | | deprivation (t44) |
| phaABCZ1Z2 | WT | 0.076 ± 0.0043 | 0.0079 ± 0.00045 | batch culture | after 24 h |
| | | | | | nitrogen |
| | | | | | deprivation (t44) |
| phaABCZ1Z2 | ΔaacS | 0.36 ± 0.0083 | 0.038 ± 0.00087 | batch culture | mid-log (t20.5) |
| phaABCZ1Z2 | ΔaacS | 4.91 ± 1.11 | 0.51 ± 0.12 | continuous with | early stationary |
| | | | | 1x xnitrogen | (66 h) |
| phaABCZ1Z2 | ΔaacS | 19.18 ± 0.85 | 2.00 ± 0.088 | continuous with | early stationary |
| | | | | 5x nitrogen | (71 h) |
| phaABCZ1Z2 | Δbdh | 19 | 2.5 | continuous with | Stationary (120 h) |
| | | | | 5x nitrogen | |
| phaA-aacS- | WT | background | NA | batch culture | NA |
| bdh | | levels* | | | |
| phaAB-ptb, | WT | background | NA | batch culture | NA |
| buk | | levels* | | | |

*Background levels are 3-HB titers comparable to H₂O, supernatant from OB3b WT cells or from OB3b WT cell + non-3HB related plasmid (mCherry).

Further increase in 3-HB titers might be accomplished by treating the culture supernatant with a 3-HB oligomer hydrolase. It was shown that 3-HB in the culture supernatant of an E. coli co-expressing a Ralstonia eutropha PHB operon and an extracellular Paucimonas lemoignei PHB depolymerase gene could be increased about 10 fold in the presence of a 3HB oligomer hydrolase from R. pickettii T1. In the same study a R. eutropha bdh null mutant produced up to 3.12 g/L 3-HB under anaerobic conditions, indicating potential superior 3-HB production in M. trichosporium overexpressing the phaABCZ1Z2 operon in a bdh KO or a aacS/bdh double-KO background.

Cultivation and Growth Parameters.

M. trichosporium OB3b were routinely cultured at 30° C. in a modified nitrate mineral salts (NMS) medium. Selective plates for M. trichosporium OB3b plates were supplemented with 25 μg/ml kanamycin, 50 μg/ml spectinomycin, 20 μg/ml gentamicin, whereas only 10 μg/ml kanamycin and 10 μg/ml gentamicin was used for cultivation in liquid NMS in sealed glass serum bottles with orbital shaking at 200-250 rpm. Liquid cultures were inoculated at OD600=0.1 (batch cultures) or 0.4 (continuous reactor) with plate-harvested biomass. Batch cultures were cultured as 30 ml culture volume in 160 ml sealed serum vials supplemented with 20% CH4 and 20% CO₂ in the headspace. Continuous reactor cultures were cultured as 100 ml culture volume in 160 ml borosilicate glass test tubes purchased from Kimble Kimax (Vineland, N.J.) continuously bubbled with 20% CH₄ and 2% CO₂ as described earlier. Escherichia coli Stellar (Takara Bio USA, Mountain View, Calif.) was used for cloning and plasmid propagation, and E. coli S17-1λ pir was used as the conjugation donor strain. E. coli strains were grown at 37° C. in Luria-Bertani (LB) broth supplemented with 50 ug/mL of kanamycin. Growth of E. coli or M. trichosporium OB3b was monitored by measuring the OD₆₀₀ using a spectrophotometer in a 1 ml cuvette.

Plasmid Construction and Strain Generation.

Heterologous genes reference or codon optimized sequences are depicted in Table 2.

TABLE 2

| | |
|---|---|
| | Locus_tag in ncbi reference |
| | genome or codon |
| | optimized sequence |
| Methylosinus trichosporium OB3b phaA | CQW49 17750 in CP023737.1 |
| Methylosinus trichosporium OB3b phaB | CQW49 17745 in CP023737.1 |
| Methylosinus trichosporium OB3b phaC | CQW49 10945 in CP023737.1 |
| Methylosinus trichosporium OB3b phaZ1 | CQW49 16675 in CP023737.1 |
| Methylosinus trichosporium OB3b phaZ2 | CQW49_05565 in CP023737.1 with an |
| | additional 57 bp upstream as per biocyc |
| | annotation with accession ID MTRI00769 |
| Methylosinus trichosporium OB3b aacS | CQW49 20795 in CP023737.1 |
| Methylosinus trichosporium OB3b bdh | CQW49_03370 in CP023737.1 |
| Clostridium butyricum ptb | ATGTCGAAGAACTTCGACGATCTCTTCT |
| (SEQ ID NO: 1) | CGCGCCTCCAGGAGGTGGAGACGAAGA |
| | AGGTCGCCGTCGCCGTCGCGCAGGACG |
| | AACCGGTCCTGGAAGCCGTCAAGGAGG |
| | CCAACGAGAAGGGCATCGCGAACGCGG |
| | TCCTCGTCGGCGACAAGGACAAGATCCA |
| | TGAGATCGCCAAGAAGATCGACATGGA |
| | CCTCACGAAGTTCGAAATCATGGACGTG |
| | AAGGACCCGAAGAAGGCGACCATGGAG |
| | GCCGTCAAGCTCGTCTCGTCCGGCAATG |
| | CCGACATGCTCATGAAGGGCCTCATCGA |
| | TACGGCCACCTTCCTGCGCTCGGTGCTG |
| | AATAAGGAAGTCGGCCTGCGCACCGGC |
| | AAGGTGATGTCGCATGTCTCGGTCTTCG |
| | AGATCGAAGGCTGGGATCGCCTGTTCTT |
| | CCTGACCGATGTCGCGTTCAATACCTAT |
| | CCGGAACTGAAGGACAAGGTGACCATC |
| | ATCAACAATGCGGTCTCGGTGGCCCATG |
| | CCTGCGGCCTGGATATGCCGAAGGTCGG |
| | CGTCGTGTGCCCGGTGGAAGTCGTGAAT |
| | CCGAACATGCCGTCCACGGTCGACGCGG |
| | CCCTGCTCGCGAAGATGTCCGACCGCGG |
| | CCAGTTCAAGGGCTGCGTGGTCGACGGC |
| | CCGTTCGCCCTCGACAACGCCATCAGCC |
| | TGGAGGCGGCCGAGCATAAGGGCGTGA |
| | AGGGCGAAGTGGCCGGCCAGGCGGACA |
| | TCCTGGTCATGCCGAACATCGAAACCGG |
| | CAATGTGATGTATAAGACGCTCACCTAT |
| | TTCGCCCCGGCGAAGAACGGCTGCCTGC |
| | TCGTCGGCACCTCCGCCCCCGCCATCCT |
| | GACGTCGCGCGCCGATACGTTCGAGACC |
| | AAGGTCAACTCCATCGCGCTGGCGGCCC |
| | TCGTGGCGGCCAAGAATAAGTGA |
| Clostridium butyricum buk | ATGGCGTATAAGCTCCTGATCATCAACC |
| (SEQ ID NO: 2) | CGGGCAGCACGTCGACGAAGATCGGCG |
| | TGTACGAGGACGAGAAGGAACTGTTCG |
| | AGGAAACCCTCCGCCACACGAATGAAG |
| | AGCTGAAGCAGTTCGATGCCATCTTCGA |
| | CCAGTTCCAGTTCCGCAAGGACGTCATC |
| | CTCAAGGTGCTCAGCGAAAAGAACTTCG |
| | ACATCAAGACGCTCTCCGCCGTCGTGGG |
| | CCGCGGCGGCATGCTCAAGCCGGTCGA |
| | GGGCGGCACGTACGCGGTGAACGACGC |
| | CATGGTCGAGGACCTGAAGGTGGGCGT |
| | GCAGGGCCCGCATGCCTCCAATCTGGGC |
| | GGCATCCTCGCCCGCTCGATCGCGGACG |
| | AGATCGGCGTGCCCTCCTTCATCGTGGA |
| | CCCGGTGGTCACGGACGAGCTCGCGGA |
| | CGTGGCGCGCCTGTCGGGCACGCCCGAC |
| | ATCCCGCGCAAGAGCAAGTTCCATGCCC |
| | TCAATCAGAAGGCCGTGGCCAAGCGCT |
| | ACGGCAAGGAGTCCGGCAAGGGCTATG |
| | AGAACCTGAACCTGGTGGTCGTGCACAT |
| | GGGCGGCGGCGTCTCCGTGGGCGCGCA |
| | CAACCATGGCAAGGTGGTCGACGTGAA |
| | CAATGCGCTCGACGGCGACGGCCCGTTC |
| | TCGCCCGAGCGCGCGGGCTCCGTGCCGG |
| | CCGGCGACCTCATCAAGATGTGCTTCTC |
| | CGGCAAGTACTCGGAGTCGGAGGTGTAT |
| | AGCAAGATCGTGGGCAAGGGCGGCTTC |
| | GTCGGCTATCTGAATACCAATGACGTCA |
| | AGGGCACCATCGATAAGATGGAGGCCG |
| | GCGATAAGGAATGCGAGAATATCTATA |
| | AGGCCTTCCTGTACCAGATCACGAAGGC |
| | CATCGGCGAGATGTCCGCCGCGCTCAAC |
| | GGCAAGGTGGATCAGATCGTGCTCACG |
| | GGCGGCATCGCGTATTCCCCGACCCTCG |
| | TGCCGGACCTCAAGGCGAATGTGGAGT |
| | GGATCGCCCCGGTGACGGTGTACCCCGG |
| | CGAGGACGAGCTCCTGGCCCTGGCGCA |
| | GGGCGCCATCCGCGTCCTCGACGGCGAA |
| | GAGAAGGCGAAGGTCTACTAA |

TABLE 3
lists intergenic RBS sequences where the STOP codon is depicted in light
blue, the predicted RBS depicted in purple and the START codon is
depicted in red font.

| | genes | | |
|---|---|---|---|
| | up/ | | |
| Operon | downstream | RBS origin | RBS sequence |
| phaA/B/ | Promoter/ | endogenous | TGCTTCGAACTTCCTCGTCTGCTTCTCGAATCG |
| | phaA | OB3b | CATGTGCGCGTCGCGATTTCATGAGCAAATTTA |
| | (SEQ ID | upstream | GCCGTTTTTGCGCGGCGCGGCGATGCGAGAGA |
| | NO: 3) | of mxaF | TTTTCCGTCTTTATTGCATGTTTTTCCCGTCCGA |
| | | | CATGACCGATAAATCATGCAGACAGAGCAACG |
| | | | CGGCTAAACTTATATGCTCGCGGTCCACCCATT |
| | | | CCGTCATCGAGCTCCCCAGTGGAGAAGGGATG |
| | | | GACGCAAAAACCAAGACGGTTTCGGACCACAC |
| | | | GACCGTCAAATCTATTTTTACGAGGCGACGAA |
| | | | AGCGCTCGACGAAAGCGCCACGAGGCGCGTGC |
| | | | GCCCGTCGCAGGAGGACTCGTCGATG |
| ptb/buk | phaA/B | endogenous | TAGAGCGCTTTCCGATCGAACGGCATCGTTCG |
| | (SEQ ID | OB3b | ATCGACCAGAATTCGCTCAAACGAAAAATGCC |
| | NO: 4) | intergenic | AGAGCGTGATGCGATCCAATCGGATCGGATCA |
| | | region | CGCTCTAGATATCGGACCGCGAGCCTTCGAGG |
| | | between | CTCGCGGTCCGGCTTTTTGACATTCGATGAACi |
| | | phaA and | GAGGACGAGGTG |
| | | phaB | |
| | phaB/ptb | Consensus | TGAAGGAGGTTAGAATTCATG |
| | (SEQ ID | RBS + 9 bp | |
| | NO: 5) | | |
| | ptb/buk | C. | TAGTAGAATATTAAATGTAAACTTTAGGATAT |
| | (SEQ ID | butyricum | AATTCTTGGGAATTTATATTAAATCACAGTGTA |
| | NO: 6) | intergenic | GATAGATTTTAATGAAAATATGGAGGAAAGA |
| | | region | AAATG |
| | | between | |
| | | ptb and buk | |
| phaA/B/ | Promoter/ | endogenous | TGCTTCGAACTTCCTCGTCTGCTTCTCGAATCG |
| C/Z1/Z2 | phaA | OB3b | CATGTGCGCGTCGCGATTTCATGAGCAAATTTA |
| | (SEQ ID | upstream | GCCGTTTTTGCGCGGCGCGGCGATGCGAGAGAT |
| | NO: 7) | of mxaF | TTTCCGTCTTTATTGCATGTTTTTCCCGTCCGA |
| | | | CATGACCGATAAATCATGCAGACAGAGCAACG |
| | | | CGGCTAAACTTATATGCTCGCGGTCCACCCATT |
| | | | CCGTCATCGAGCTCCCCAGTGGAGAAGGGATG |
| | | | GACGCAAAAACCAAGACGGTTTCGGACCACAC |
| | | | GACCGTCAAATCTATTTTTACGAGGCGACGAA |
| | | | AGCGCTCGACGAAAGCGCCACGAGGCGCGTGC |
| | | | GCCCGTCGCAGGAGGACTCGTCGATG |
| | phaA/B | endogenous | TAGAGCGCTTTCCGATCGAACGGCATCGTTCG |
| | (SEQ ID | OB3b | ATCGACCAGAATTCGCTCAAACGAAAAATGCC |
| | NO: 8) | intergenic | AGAGCGTGATGCGATCCAATCGGATCGGATCA |
| | | region | CGCTCTAGATATCGGACCGCGAGCCTTCGAGG |
| | | between | CTCGCGGTCCGGCTTTTTGACATTCGATGAAG |
| | | phaA and | GAGGACGAGGTG |
| | | phaB | |
| | phaB/C | Consensus | TGAAGGAGGTTTTTTATG |
| | (SEQ ID | RBS + 6bp | |
| | NO: 9) | | |
| | phaC/Z1 | designed | TGACCGAGAGGAAGGGACGTTACCTAATG |
| | (SEQ ID | with RBS | |
| | NO: 10) | calculator | |
| | phaZ1/Z2 | Consensus | TGAAGGAGGTTATAAAAAATG |
| | (SEQ ID | RBS + 9 bp | |
| | NO: 11) | | |
| phaA/aa | Promoter/ | same as | |
| cS/bdh | phaA | above | |
| | aacS/bdh | Consensus | TGAAGGAGGTTATAAAAATG |
| | (SEQ ID | RBS + 9 bp | |
| | NO: 12) | | |
| phaA/B/ | Promoter/ | endogenou | TGCTTCGAACTTCCTCGTCTGCTTCTCGAATCG |
| ptbOpt/b | phaA | s OB3b | CATGTGCGCGTCGCGATTTCATGAGCAAATTTA |
| ukOpt | (SEQ ID | upstream | GCCGTTTTTGCGCGGCGCGGCGATGCGAGAGAT |
| | NO: 13) | of mxaF | TTTCCGTCTTTATTGCATGTTTTTCCCGTCCGA |
| | | | CATGACCGATAAATCATGCAGACAGAGCAACGC |
| | | | GGCTAAACTTATATGCTCGCGGTCCACCCATT |
| | | | CCGTCATCGAGCTCCCCAGTGGAGAAGGGATG |
| | | | GACGCAAAAACCAAGACGGTTTCGGACCACAC |
| | | | GACCGTCAAATCTATTTTTACGAGGCGACGAA |
| | | | AGCGCTCGACGAAAGCGCCACGAGGCGCGTGC |
| | | | GCCCGTCGCAGGAGGACTCGTCGATG |
| | phaA/B | endogenous | TAGAGCGCTTTCCGATCGAACGGCATCGTTCG |
| | (SEQ ID | OB3b | ATCGACCAGAATTCGCTCAAACGAAAAATGCC |
| | NO: 14) | intergenic | AGAGCGTGATGCGATCCAATCGGATCGGATCA |
| | | region | CGCTCTAGATATCGGACCGCGAGCCTTCGAGG |
| | | between | CTCGCGGTCCGGCTTTTTGACATTCGATGAAG |
| | | phaA and | GAGGACGAGGTG |
| | | phaB | |
| | phaB/ptb | Consensus | TGAAGGAGGTTAGAATTCATG |
| | (SEQ ID | RBS + 9 bp | |
| | NO: 15) | | |
| | ptb/buk | Consensus | TGAAGGAGGTTATAAAAATG |
| | (SEQ ID | RBS + 9 bp | |
| | NO: 16) | | |

Strains and plasmids used in this study are presented in Table 4.

Table 4 lists strains and plasmids used herein.

| Strains | Genotype/Description | References |
|---|---|---|
| Methylosinus trichosporium | Wild-type | ATCC |
| OB3b | | |
| Methylosinus trichosporium | aacS (gene with locus tag | This study |
| OB3b ΔaacS | CQW49_20795 in | |
| | CP023737.1) knock out | |
| Methylosinus trichosporium | bdh (gene with locus tag | This study |
| OB3b Δbdh | CQW49_03370 in | |
| | CP023737.1) knock out | |
| E. coli Stellar (an E. coli | F-, endA1, supE44, thi-1, | Takara Bio USA, Inc. |
| HST08 strain) | recA1, relA1, gyrA96, phoA, | |
| | Φ80d lacZΔ M15, | |
| | Δ(lacZYA-argF) U169, | |
| | Δ(mrr-hsdRMS-mcrBC), | |
| | ΔmcrA, λ- | |
| E. coli S17-1 λpir | Tpr Smr recA thi pro hsd(r - | ATCC |
| | m+)RP4-2-Tc::Mu::Km Tn7 | |
| | λpir | |

Plasmids

| | | |
|---|---|---|
| pIPRJ016 | M. trichosporium OB3b | This study |
| | phaZ1-phaZ2 inserted | |
| | downstream of PmxaF using | |
| | InFusion assembly | |
| pIPRJ017 | M. trichosporium OB3b | This study |
| | phaA-aacS-bdh inserted | |
| | downstream of PmxaF using | |
| | InFusion assembly | |
| pIPRJ029 | M. trichosporium OB3b | This study |
| | phaA-phaB and codon | |
| | optimized | |
| | Clostridium butyricum | |
| | ptb and buk | |
| | inserted downstream of | |
| | PmxaF using InFusion | |
| | assembly | |
| pIPRJ032 | M. trichosporium OB3b | This study |
| | phaA-phaB-phaC-phaZ1- | |
| | phaZ2 inserted downstream | |
| | of PmxaF using InFusion | |
| | assembly | |

Plasmids for heterologous gene expression were constructed using 5× InFusion HD Enzyme Mix from Takara Bio USA, Inc. (Mountain View, Calif.) following the manufacturers protocol. Polymerase chain reactions were performed using Q5 High-Fidelity Polymerase from New England Biolabs and primers (Table 5) purchased from Integrated DNA Technologies (Coralville, Iowa). Heterologous genes under the control of the M. trichosporium OB3b mxaF (locus tag CQW49_RS14460 in NCBI Reference Sequence: NZ_CP023737.1) upstream region (PmxaF) were inserted into a modified IncP-containing pAWP78 backbone. Final constructs were confirmed by sequence analysis (Genewiz, South Plainfield, N.J.).

TABLE 5
Primers. Lowercase denotes homologous sequence for InFusion assembly,
lowercase & bold are additional spacer (e.g. RBS sequences)
added during assembly.
Cloning

| Name | Primer sequence (5′ to 3′) | |
|---|---|---|
| oCAH033 (SEQ ID NO: 17) | F: TTGTCGGGAAGATGCGTG | pAWP78 backbone |
| oCAH026 (SEQ ID NO: 18) | R: CAGCTCACTCAAAGGCGG | |
| oRJ117 (SEQ ID NO: 19) | F: | rrnBTlT2 |
| | AGGCATCAAATAAAACGAAAG | downstream of GOI |
| | GCTCAGTCGAAAG | |
| OCAH032 (SEQ ID NO: 20) | R: | |
| | gaaggatcagatcacgcatcttcccgacaaCAT | |
| | CCGTCAGGATGGCCT | |
| oRJ078 (SEQ ID NO: 21) | F: | upstream region of |
| | cgtattaccgcctttgagtgagctgTGCTTCG | OB3b mxaF (PmxaF) |
| | AACTTCCTCGTCTG | upstream of GOI |
| oRJ079 (SEQ ID NO: 22) | R: CGACGAGTCCTCCTGCGAC | |
| oRJ135 (SEQ ID NO: 23) | F: | OB3b phaZ1 |
| | cgtcgcaggaggactcgtcgATGGATCGG | downstream of |
| | CTGTCTTACCAGCTCTATG | PmxaF |
| oRJ133 (SEQ ID NO: 24) | R: TCAGCCGCCCGCCTGCTC | |
| oRJ109 (SEQ ID NO: 25) | F: | OB 3 b phaZ2 |
| | gagggtggagcaggcgggcggctgaaggaggt | downstream of OB3b |
| | tataaaaaATGGCATGGGGCTTGC | phaZ1 |
| | TG | |
| oRJ004 (SEQ ID NO: 26) | R: | |
| | tttcgttttatttgatgcctCTACACGCTGTT | |
| | CTGGATATGG | |
| oRJ101 (SEQ ID NO: 27) | F: | OB3b phaA upstream |
| | cgtcgcaggaggactcgtcgATGACGACG | of OB3b aacS |
| | GAAATCGTG | |
| oRJ140 (SEQ ID NO: 28) | R:CTCGTCCTCCTTCATCGAATG | |
| oRJ136 (SEQ ID NO: 29) | F: | OB3b aacS |
| | ttgacattcgatgaaggaggacgagATGCCG | downstream of OB3b |
| | ATCTGGAAGCCC | phaA |
| oRJ137 (SEQ ID NO: 30) | R:TCACTTCCCGGACAGCTG | |
| oRJ138 (SEQ ID NO: 31) | tctgccgcagctgtccgggaagtgaaggag | OB3b bdh |
| | gttataaaaATGTCTCTCGCCAAACGC | downstream of OB3b |
| | AAC | aacS |
| oRJ139 (SEQ ID NO: 32) | gagcctttcgttttatttgatgcctTCA | |
| | CGCCGCCGTCCAGCC | |
| oRJ101 (SEQ ID NO: 33) | F: | OB 3 b phaAB |
| | cgtcgcaggaggactcgtcgATGACGACG | upstream of codon- |
| | GAAATCGTG | optimized ptb/OB3b |
| oRJ102 (SEQ ID NO: 34) | R:TCAGGTCAGATACTGGCC | phaC |
| oRJ199 (SEQ ID NO: 35) | F: | OB3b phaC |
| | gcggccagtatctgacctgaagg | downstream of OB3b |
| | aggttttttATGACCGCAGGACGCCGC | phaAB |
| oRJ201 (SEQ ID NO: 36) | R: | |
| | gtaacctcccttcctctcggTCACGCCCTC | |
| | ACGCGCAC | |
| oRJ202 | RBS US ofphaZ1 in pIPRJ015 FWD | OB3b phaZ1Z2 |
| oRJ004 (SEQ ID NO: 37) | R: | downstream of OB3b |
| | tttcgttttatttgatgcctCTACACGCTGTT | phaC |
| | CTGGATATGG | |

Sequencing

| Name | Primer sequence (5′ to 3′) | Gene to sequence |
|---|---|---|
| OCAH1225 (SEQ ID NO: 38) | F: CTGTGGATAACCGTATTACCG | PmxaF + 5′ end of |
| | | GOI |
| OCAH048 (SEQ ID NO: 39) | R: | |
| | GGCGGCTTTGTTGAATAAATCGA | rrnBT1T2 + 3′ end |
| | A | of GOI OB3b |
| oRJ146 (SEQ ID NO: 40) | R: ATCAGCACCATGGATTCG | phaZ1/PmxaF |
| oRJ133 (SEQ ID NO: 41) | R: TCAGCCGCCCGCCTGCTC | OB3b phaZ1 |
| oRJ238 (SEQ ID NO: 42) | R: CGTCATACATCATGCGGCTC | OB3b phaZ2/Z1 |
| oRJ145 (SEQ ID NO: 43) | F: ATTATCTGCAAGAGGGCG | OB3b phaZ2/Z1 |
| oRJ144 (SEQ ID NO: 44) | R: AAGCAGGAGACGATGTCG | OB3b aacS |
| oRJ149 (SEQ ID NO: 45) | F: CGCTCAAACGAAAAATGCC | OB3b aacS/phaB |
| oRJ148 (SEQ ID NO: 46) | F: CTCGACAAGCACATCATC | OB3b phaC/ |
| | | C. butyricum ptb |
| oRJ230 (SEQ ID NO: 47) | R: ATGTGCACGACCACCAGG | C. butyricum |
| | | buk/ptb |
| oRJ237 (SEQ ID NO: 48) | R: GACATTATAGCCTGCCGCCT | OB3b phaB/A |
| oRJ204 (SEQ ID NO: 49) | R: GCGGAAAAGCGCTTGTCG | OB3b phaC |
| oRJ203 (SEQ ID NO: 50) | F: GCAGCGAATTGGCGAGTAAT | OB3b phaC |
| oRJ206 (SEQ ID NO: 51) | R: GATGAACACCTGCATGTCGC | OB3b phaC |
| oRJ205 (SEQ ID NO: 52) | F: CTCGACGCGGTGCAGAAG | OB3b phaC |

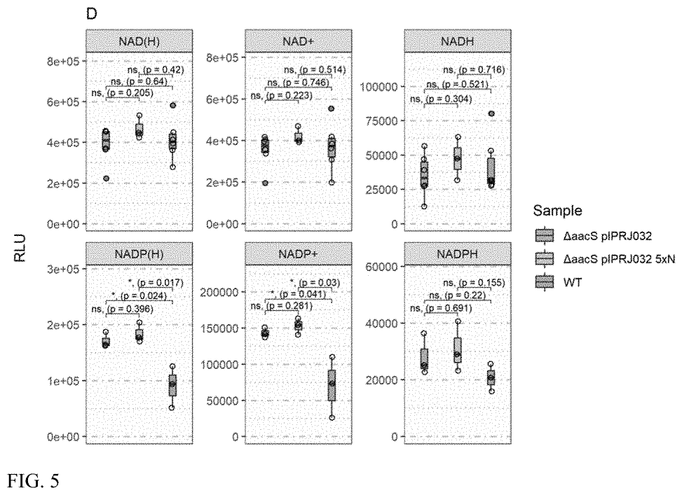

Plasmid constructs were transformed into M. trichosporium OB3b via conjugation as previously described with the following modifications: an overflowing 10 ul inoculation loop of M. trichosporium OB3b was spread on NMS mating plates and grown overnight. An equal volume of Escherichia coli S17-1 λpir donor biomass containing the vector of interest was then added to the plate, and the resulting mixture was incubated at 30° C. for 2 days. The resulting biomass was collected, resuspended in NMS liquid medium and a fraction was plated on selective NMS plates supplemented with 10 μg/ml nalidixic acid to remove S17 donor cells. Stock solutions of nalidixic acid were prepared at 10 mg/ml in H₂O. After adjusting the pH to 11, the solution was sterile filtered and aliquots were stored at −20° C. Genomic knock-outs of M. trichosporium OB3b cells were generated by removing the coding sequence via homologous recombination induced by introduction of a linear DNA fragment via electroporation as described previously. The linear DNA fragments, including 1 kb DNA regions upstream and downstream of the gene of interest and a gentamicin resistance cassette, were constructed via fusion PCR as described previously, whereas overlapping ends were only added to the homology region to ease reusability of the selection marker. If fusion PCR was not successful the respective linear DNA fragment was ordered as a clonal gene from Twist Biosciences, San Francisco, Calif., and amplified from the vector via high fidelity PCR. KO background strains were confirmed via high fidelity PCR for specific target regions (see FIG. 5 for schematic) using either boiled cells or gDNA as template. The resulting product was analyzed visually via strand separation on an agarose gel, fragments of the expected length were purified, and the sequence identity was confirmed by sequence analysis (Genewiz, South Plainfield, N.J.).

Relative PHB Quantification Via Staining Cells with Nile Red.

Cellular PHB concentration was quantified relative to a WT sample by staining WT and mutant strain with nile red. Cells equivalent to an OD600 of 5 were harvested and the resulting pellet was resuspended in 900 μl NMS+100μ nile red solution (250 μg/ml nile red in dimethyl sulfoxide). After 15 min incubation, 200 μl aliquots were assayed for fluorescence at excitation (535 nm) and emission (605 nm) wavelength at a plate reader (Infinite M PLEX by Tecan Trading AG, Switzerland).

Acid Quantification in the Supernatant

To quantify acid levels in culture supernatant, an aliquot of culture broth was taken, filtered through a 0.2 μm syringe filter (SY25GN by mdi Membrane Technologies INC., Harrisburg, Pa.). The resulting clear supernatant was either analyzed by HPLC or 3-HB levels were determined using the β-Hydroxybutyrate (Ketone Body) Colorimetric Assay Kit by Cayman Chemical, Ann Arbor, Mich. following the manufacturers protocol. According to the manufacturer, the kit only detects the 3-HB D-isoform—which appears physiologically in humans and other animals—due to enzyme stereo-specificity. Succinic, lactic, formic and acetic acid levels were quantified via HPLC as described earlier.

Dry Cell Weight Measurement

Microbial cultures (7-90 ml) were centrifuged to collect biomass in tubes of known weight. Cell pellets were dried in those tubes in a 45° C. vacuum oven for at least 48 h prior to weighing them again. DCW was determined by subtracting the empty tube weight from the tube weight plus dried biomass and dividing by the volume of biomass harvested. The calculated DCW (mg/L) was plotted over OD600 and a linear model using the lm( ) function in R yielded a linear regression curve with y-intercept=−8.058, slope=228.629, p-value: 2.2×10⁻¹⁶.

NAD+/NADH Quantification.

To determine NAD+/NADH or NADP+/NADPH levels in M. trichosporium cells, mid-log culture was harvested to yield 3.33×10⁷ cells/well of a 96 well plate and analyzed with the NAD/NADH-Glo™ or NADP/NADPH-Go™ kit by Promega Corporation (Madison, Wis.). The cell pellet was resuspended in 300 ul high pH Bicarbonate Buffer+1% DTAB and either processed immediately or, if necessary, stored at −80° C. 100 μl samples were acid/base treated, neutralized and 50 μl of the final reagent mixture was analyzed with the detection reagent following the manufacturers protocol. Relative luminescence units were determined after 60 min using white, flat bottom 96-well microplates procured from Tecan Trading AG, Switzerland, in an Infinite M PLEX plate reader (Tecan Trading AG, Switzerland).

RNA Isolation and RT-qPCR

To compare transcript levels to the wild-type levels, 1 ml of mid-log culture (corresponding to an OD600 between 3.5 and 10) from the continuous bioreactor was harvested. RNA levels were stabilized by resuspending the culture in 1 ml RNAlater (Thermo Fisher Scientific Inc., Waltham, Mass.) and samples were stored at −80° C. till further processing. RNA was extracted using the RNeasy Mini Kit (Qiagen, Hilden, Germany) along with on-column DNase Digestion using the RNAse-Free DNase Set (Qiagen, Hilden, Germany) following the manufacturer's procedures. RNA levels were determined using the iTaq™ Universal SYBR® Green One-Step Kit (Bio-Rad, Hercules, Calif.) in a QuantStudio™ 3 Real-Time PCR System (Applied Biosystems, Waltham, Mass.). Relative expression levels of target genes in a sample relative to a wild-type control were calculated using the ΔΔCt method with the M. trichosporium OB3b RNA polymerase sigma factor rpoD housekeeping gene (locus tag CQW49_RS04780 in CP023737.1) as reference gene.

The foregoing disclosure has been set forth merely to illustrate the invention and is not intended to be limiting.

We claim:

1. A non-naturally occurring organism capable of converting gases selected from the group consisting essentially of CO₂, stranded natural gas, flue gas, biogas, and landfill gas to produce 3-hydroxybutyrate.

2. The non-naturally occurring organism of claim 1 wherein the organism is a methanotrophic bacteria.

3. The non-naturally occurring organism of claim 1 comprising an M. trichosporium OB3b strain.

4. The non-naturally occurring organism of claim 1 wherein the organism is genetically engineered to overexpress PHB depolymerase and also lacks an acetoacetyl-CoA synthetase (AACS) pathway.

5. The non-naturally occurring organism of claim 1 wherein the production of 3-hydroxyburtyrate is up to 19 mM.

6. The non-naturally occurring organism of claim 1 comprising a gene having a sequence identify greater than 70% identical to SEQ ID NO: 1 encoding for a non-naturally occurring phosphate butyryltransferase.

7. The non-naturally occurring organism of claim 1 comprising a gene having a sequence identify greater than 70% identical to SEQ ID NO: 2 encoding for a non-naturally occurring butyrate kinase.

8. The non-naturally occurring organism of claim 1 comprising a member of M. trichosporium sp.

9. The non-naturally occurring organism of claim 1 comprising strain M. trichosporium OB3b.

10. A method for making 3-hydroxybutyrate comprising the step of contacting a non-naturally occurring organism with gases selected from the group consisting essentially of CO₂, stranded natural gas, flue gas, biogas, and landfill gas.

11. The method of claim 10 wherein the non-naturally occurring organism is a methanotrophic bacteria.

12. The method of claim 10 wherein the non-naturally occurring organism comprises a member of M. trichosporium sp.

13. The method of claim 10 wherein the non-naturally occurring organism is a M. trichosporium OB3b strain.

14. The method of claim 10 wherein the non-naturally occurring organism is genetically engineered to overexpress PHB depolymerase and also lacks a AACS pathway.

15. The method of claim 10 wherein the production of 3-hydroxyburtyrate is up to 19 mM.

16. The method of claim 10 wherein the method comprises using a gene having a sequence identify greater than 70% identical to SEQ ID NO: 1 encoding for a non-naturally occurring phosphate butyryltransferase.

17. The method of claim 10 wherein the method comprises using a gene having a gene sequence identify greater than 70% identical to SEQ ID NO: 2 encoding for a non-naturally occurring butyrate kinase.

18. A system for the conversion of gases selected from the group consisting essentially of CO₂, stranded natural gas, flue gas, biogas, and landfill gas to produce 3-hydroxybutyrate using a non-naturally occurring organism capable of converting the gasses to produce 3-hydroxybutyrate.

19. The system of claim 18 comprising a non-naturally occurring member of M. trichosporium sp. comprising a gene having a sequence identify greater than 70% identical to SEQ ID NO: 1 encoding for a non-naturally occurring phosphate butyryltransferase and further comprising a gene having a sequence identify greater than 70% identical to SEQ ID NO: 2 encoding for a non-naturally occurring butyrate kinase.

20. The system of claim 18 wherein the system is capable of the production of 3-hydroxyburtyrate up to a concentration of 19 mM.


SEQUENCE LISTING (52 sequences)

SEQ ID NO: 1 - DNA, 909 bp, Clostridium butyricum
atgtcgaagaacttcgacgatctcttctcgcgcctccaggaggtggagacgaagaaggtcgccgtcgccgtcgcgcaggacgaaccggtcctggaagccgtcaaggaggccaacgagaagggcatcgcgaacgcggtcctcgtcggcgacaaggacaagatccatgagatcgccaagaagatcgacatggacctcacgaagttcgaaatcatggacgtgaaggacccgaagaaggcgaccatggaggccgtcaagctcgtctcgtccggcaatgccgacatgctcatgaagggcctcatcgatacggccaccttcctgcgctcggtgctgaataaggaagtcggcctgcgcaccggcaaggtgatgtcgcatgtctcggtcttcgagatcgaaggctgggatcgcctgttcttcctgaccgatgtcgcgttcaatacctatccggaactgaaggacaaggtgaccatcatcaacaatgcggtctcggtggcccatgcctgcggcctggatatgccgaaggtcggcgtcgtgtgcccggtggaagtcgtgaatccgaacatgccgtccacggtcgacgcggccctgctcgcgaagatgtccgaccgcggccagttcaagggctgcgtggtcgacggcccgttcgccctcgacaacgccatcagcctggaggcggccgagcataagggcgtgaagggcgaagtggccggccaggcggacatcctggtcatgccgaacatcgaaaccggcaatgtgatgtataagacgctcacctatttcgccccggcgaagaacggctgcctgctcgtcggcacctccgcccccgccatcctgacgtcgcgcgccgatacgttcgagaccaaggtcaactccatcgcgctggcggccctcgtggcggccaagaataagtga

SEQ ID NO: 2 - DNA, 1068 bp, Clostridium butyricum
atggcgtataagctcctgatcatcaacccgggcagcacgtcgacgaagatcggcgtgtacgaggacgagaaggaactgttcgaggaaaccctccgccacacgaatgaagagctgaagcagttcgatgccatcttcgaccagttccagttccgcaaggacgtcatcctcaaggtgctcagcgaaaagaacttcgacatcaagacgctctccgccgtcgtgggccgcggcggcatgctcaagccggtcgagggcggcacgtacgcggtgaacgacgccatggtcgaggacctgaaggtgggcgtgcagggcccgcatgcctccaatctgggcggcatcctcgcccgctcgatcgcggacgagatcggcgtgccctccttcatcgtggacccggtggtcacggacgagctcgcggacgtggcgcgcctgtcgggcacgcccgacatcccgcgcaagagcaagttccatgccctcaatcagaaggccgtggccaagcgctacggcaaggagtccggcaagggctatgagaacctgaacctggtggtcgtgcacatgggcggcggcgtctccgtgggcgcgcacaaccatggcaaggtggtcgacgtgaacaatgcgctcgacggcgacggcccgttctcgcccgagcgcgcgggctccgtgccggccggcgacctcatcaagatgtgcttctccggcaagtactcggagtcggaggtgtatagcaagatcgtgggcaagggcggcttcgtcggctatctgaataccaatgacgtcaagggcaccatcgataagatggaggccggcgataaggaatgcgagaatatctataaggccttcctgtaccagatcacgaaggccatcggcgagatgtccgccgcgctcaacggcaaggtggatcagatcgtgctcacgggcggcatcgcgtattccccgaccctcgtgccggacctcaaggcgaatgtggagtggatcgccccggtgacggtgtaccccggcgaggacgagctcctggccctggcgcagggcgccatccgcgtcctcgacggcgaagagaaggcgaaggtctactaa

SEQ ID NO: 3 - DNA, 351 bp, Artificial Sequence
  Promoter/phaA; endogenous OB3b upstream of mxaF
tgcttcgaacttcctcgtctgcttctcgaatcgcatgtgcgcgtcgcgatttcatgagcaaatttagccgtttttgcgcggcgcggcgatgcgagagattttccgtctttattgcatgtttttcccgtccgacatgaccgataaatcatgcagacagagcaacgcggctaaacttatatgctcgcggtccacccattccgtcatcgagctccccagtggagaagggatggacgcaaaaaccaagacggtttcggaccacacgaccgtcaaatctatttttacgaggcgacgaaagcgctcgacgaaagcgccacgaggcgcgtgcgcccgtcgcaggaggactcgtcgatg

SEQ ID NO: 4 - DNA, 172 bp, Artificial Sequence
  phaA/B; endogenous OB3b intergenic region between phaA and phaB
tagagcgctttccgatcgaacggcatcgttcgatcgaccagaattcgctcaaacgaaaaatgccagagcgtgatgcgatccaatcggatcggatcacgctctagatatcggaccgcgagccttcgaggctcgcggtccggctttttgacattcgatgaaggaggacgaggtg

SEQ ID NO: 5 - DNA, 21 bp, Artificial Sequence
  phaB/ptb; Consensus RBS + 9 bp
tgaaggaggttagaattcatg

SEQ ID NO: 6 - DNA, 101 bp, Artificial Sequence
  ptb/buk; C. butyricum intergenic region between ptb and buk
tagtagaatattaaatgtaaactttaggatataattcttgggaatttatattaaatcacagtgtagatagattttaatgaaaatatggaggaaagaaaatg

SEQ ID NO: 7 - DNA, 351 bp, Artificial Sequence; identical to SEQ ID NO: 3
  Promoter/phaA; endogenous OB3b upstream of mxaF

SEQ ID NO: 8 - DNA, 172 bp, Artificial Sequence; identical to SEQ ID NO: 4
  phaA/B; endogenous OB3b intergenic region between phaA and phaB

SEQ ID NO: 9 - DNA, 18 bp, Artificial Sequence
  phaB/C; Consensus RBS + 6 bp
tgaaggaggttttttatg

SEQ ID NO: 10 - DNA, 29 bp, Artificial Sequence
  phaC/Z1; designed with RBS calculator
tgaccgagaggaagggaggttacctaatg

SEQ ID NO: 11 - DNA, 21 bp, Artificial Sequence
  phaZ1/Z2; Consensus RBS + 9 bp
tgaaggaggttataaaaaatg

SEQ ID NO: 12 - DNA, 20 bp, Artificial Sequence
  aacS/bdh; Consensus RBS + 9 bp
tgaaggaggttataaaaatg

SEQ ID NO: 13 - DNA, 351 bp, Artificial Sequence; identical to SEQ ID NO: 3
  Promoter/phaA; endogenous OB3b upstream of mxaF

SEQ ID NO: 14 - DNA, 172 bp, Artificial Sequence; identical to SEQ ID NO: 4
  phaA/B; endogenous OB3b intergenic region between phaA and phaB

SEQ ID NO: 15 - DNA, 21 bp, Artificial Sequence
  phaB/ptb; Consensus RBS + 9 bp
tgaaggaggttagaattcatg

SEQ ID NO: 16 - DNA, 20 bp, Artificial Sequence
  ptb/buk; Consensus RBS + 9 bp
tgaaggaggttataaaaatg

SEQ ID NO: 17 - DNA, 18 bp, Artificial Sequence
  Primer CAH033
ttgtcgggaagatgcgtg

SEQ ID NO: 18 - DNA, 18 bp, Artificial Sequence
  Primer CAH026
cagctcactcaaaggcgg

SEQ ID NO: 19 - DNA, 34 bp, Artificial Sequence
  Primer RJ117
aggcatcaaataaaacgaaaggctcagtcgaaag

SEQ ID NO: 20 - DNA, 48 bp, Artificial Sequence
  Primer CAH032
gaaggatcagatcacgcatcttcccgacaacatccgtcaggatggcct

SEQ ID NO: 21 - DNA, 46 bp, Artificial Sequence
  Primer RJ078
cgtattaccgcctttgagtgagctgtgcttcgaacttcctcgtctg

SEQ ID NO: 22 - DNA, 19 bp, Artificial Sequence
  Primer RJ079
cgacgagtcctcctgcgac

SEQ ID NO: 23 - DNA, 48 bp, Artificial Sequence
  Primer RJ135
cgtcgcaggaggactcgtcgatggatcggctgtcttaccagctctatg

SEQ ID NO: 24 - DNA, 18 bp, Artificial Sequence
  Primer RJ133
tcagccgcccgcctgctc

SEQ ID NO: 25 - DNA, 58 bp, Artificial Sequence
  Primer RJ109
gagggtggagcaggcgggcggctgaaggaggttataaaaaatggcatggggcttgctg

SEQ ID NO: 26 - DNA, 42 bp, Artificial Sequence
  Primer RJ004
tttcgttttatttgatgcctctacacgctgttctggatatgg

SEQ ID NO: 27 - DNA, 38 bp, Artificial Sequence
  Primer RJ101
cgtcgcaggaggactcgtcgatgacgacggaaatcgtg

SEQ ID NO: 28 - DNA, 21 bp, Artificial Sequence
  Primer RJ140
ctcgtcctccttcatcgaatg

SEQ ID NO: 29 - DNA, 43 bp, Artificial Sequence
  Primer RJ136
ttgacattcgatgaaggaggacgagatgccgatctggaagccc

SEQ ID NO: 30 - DNA, 18 bp, Artificial Sequence
  Primer RJ137
tcacttcccggacagctg

SEQ ID NO: 31 - DNA, 60 bp, Artificial Sequence
  Primer RJ138
tctgccgcagctgtccgggaagtgaaggaggttataaaaatgtctctcgccaaacgcaac

SEQ ID NO: 32 - DNA, 43 bp, Artificial Sequence
  Primer RJ139
gagcctttcgttttatttgatgccttcacgccgccgtccagcc

SEQ ID NO: 33 - DNA, 38 bp, Artificial Sequence; identical to SEQ ID NO: 27
  Primer RJ101

SEQ ID NO: 34 - DNA, 18 bp, Artificial Sequence
  Primer RJ102
tcaggtcagatactggcc

SEQ ID NO: 35 - DNA, 50 bp, Artificial Sequence
  Primer RJ199
gcggccagtatctgacctgaaggaggttttttatgaccgcaggacgccgc

SEQ ID NO: 36 - DNA, 38 bp, Artificial Sequence
  Primer RJ201
gtaacctcccttcctctcggtcacgccctcacgcgcac

SEQ ID NO: 37 - DNA, 42 bp, Artificial Sequence; identical to SEQ ID NO: 26
  Primer RJ004

SEQ ID NO: 38 - DNA, 21 bp, Artificial Sequence
  Primer CAH1225
ctgtggataaccgtattaccg

SEQ ID NO: 39 - DNA, 24 bp, Artificial Sequence
  Primer CAH048
ggcggctttgttgaataaatcgaa

SEQ ID NO: 40 - DNA, 18 bp, Artificial Sequence
  Primer RJ146
atcagcaccatggattcg

SEQ ID NO: 41 - DNA, 18 bp, Artificial Sequence
  Primer RJ133
tcagccgcccgcctgctc

SEQ ID NO: 42 - DNA, 20 bp, Artificial Sequence
  Primer RJ238
cgtcatacatcatgcggctc

SEQ ID NO: 43 - DNA, 18 bp, Artificial Sequence
  Primer RJ145
attatctgcaagagggcg

SEQ ID NO: 44 - DNA, 18 bp, Artificial Sequence
  Primer RJ144
aagcaggagacgatgtcg

SEQ ID NO: 45 - DNA, 19 bp, Artificial Sequence
  Primer RJ149
cgctcaaacgaaaaatgcc

SEQ ID NO: 46 - DNA, 18 bp, Artificial Sequence
  Primer RJ148
ctcgacaagcacatcatc

SEQ ID NO: 47 - DNA, 18 bp, Artificial Sequence
  Primer RJ230
atgtgcacgaccaccagg

SEQ ID NO: 48 - DNA, 20 bp, Artificial Sequence
  Primer RJ237
gacattatagcctgccgcct

SEQ ID NO: 49 - DNA, 18 bp, Artificial Sequence
  Primer RJ204
gcggaaaagcgcttgtcg

SEQ ID NO: 50 - DNA, 20 bp, Artificial Sequence
  Primer RJ203
gcagcgaattggcgagtaat

SEQ ID NO: 51 - DNA, 20 bp, Artificial Sequence
  Primer RJ206
gatgaacacctgcatgtcgc

SEQ ID NO: 52 - DNA, 18 bp, Artificial Sequence
  Primer RJ205
ctcgacgcggtgcagaag